(19) United States
(12) Patent Application Publication
(10) Pub. No.: US 2023/0346207 A1
(43) Pub. Date: Nov. 2, 2023

(54) CONTROL METHOD AND CONTROL SYSTEM FOR MAGNETIC CONTROL CAPSULE ENDOSCOPIC APPARATUS

(71) Applicant: Ankon Medical Technologies?Shanghai) Co., LTD, Pilot Free Trade (CN)

(72) Inventors: Xin LIU, Shanghai (CN); Tingqi WANG, Shanghai (CN); Yang LI, Shanghai (CN); Shaobang ZHANG, Shanghai (CN)

(73) Assignees: Ankon Medical Technologies?Shanghai) Co., LTD, Shanghai, CN (CN); ANX IP HOLDING PTE. LTD., SG, SG (SG)

(21) Appl. No.: 18/026,304

(22) PCT Filed: Sep. 9, 2021
(86) PCT No.: PCT/CN2021/117337

§ 371 Date: Mar. 14, 2023

(30) Foreign Application Priority Data
Sep. 14, 2020 (CN) 202010963287.3

(51) Int. Cl. A61B 1/00; A61B 1/04

(52) U.S. Cl. CPC A61B 1/00158; A61B 1/041; A61B 1/00006

(57) ABSTRACT

The present invention provides a control method and a control system for a magnetic control capsule endoscopic apparatus. The method comprises: after the magnetic control capsule endoscopic apparatus is started, detecting a current motion mode thereof, where the motion mode comprises a vehicle-mounted mode and a normal mode; if the motion mode is configured as the normal mode, keeping a position of a magnetic ball unchanged, and directly entering an examination procedure; and if the motion mode is configured as the vehicle-mounted mode, after adjusting the position of the magnetic ball from a default position corresponding to the vehicle-mounted mode to a default position corresponding to the normal mode, entering the examination procedure. According to the method and the system, the position of the magnetic ball is adjusted, such that a plurality of application environments are adapted to, thereby improving the safety performance of the magnetic control capsule endoscopic apparatus.

### CROSS-REFERENCE OF RELATED APPLICATIONS

The application claims priority from Chinese Patent Application No. 202010963287.3, filed Sep. 14, 2020, entitled “Control Method and Control System for Magnetic Capsule Endoscopic Apparatus”, all of which are incorporated herein by reference in their entirety.

### FIELD OF INVENTION

The present invention relates to the field of communication control of medical apparatus, and more particularly to a control method and a control system for a magnetic control capsule endoscopic apparatus.

### BACKGROUND

A magnetic control capsule endoscopic apparatus is a medical device comprising: an external control device, and a capsule endoscope. The capsule endoscope integrates core devices such as a camera and a wireless transmission antenna into an ingestible capsule-shaped device that can be swallowed by a subject. During an examination, the external control device cooperates with the capsule endoscope swallowed into the subject, and the external control device controls the capsule endoscope to capture images of digestive tract of the subject and synchronously transmit the images back to the external control device, while a medical examination is carried out according to the captured images.

Due to its enormous size, the external control device is generally fixed in a specific room of a medical facility. According to FIG. 1, the external control device comprises: a base 10, an examination bed 20 installed at the base 10 and close to the lower part of the base, a driving mechanism 30 installed at the base 10 in a cantilevered mode, and a magnetic ball 40 connected to the bottom of a part of the driving mechanism 30 and positioned above the examination bed 20. The driving mechanism 30 is configured to control and adjust the position of the magnetic ball 40.

In the prior art, the magnetic ball 40 is a core component of the external control device for generating an external control magnetic field. During the examination, the magnetic ball 40 is driven to the highest and furthest point from the examination bed 20 when the device is started and set to working mode or after the device is turned off in order to facilitate using the examination bed 20 and prevent the magnetic ball 40 from being accidentally collided and affecting its performance.

However, in actual use, the external control device can be applied in a mobile carrier during a medical examination in addition to being installed in a stationary carrier, for example, in a mobile medical vehicle, as demand increases. If the position of the magnetic ball 40 is adjusted in accordance with the operation mode of the stationary carrier, the center of gravity of the entire external control device can be changed due to the high position of the magnetic ball 40 during the movement of the mobile carrier. The rise of the center of gravity can consequently indirectly affect the stability of the external control device and the mobile carrier during movement, so that both the external control device and the mobile carrier may cause safety hazards.

### SUMMARY OF THE INVENTION

To solve at least one problem that exists in the prior art, the present invention discloses a control method and a control system for a magnetic control capsule endoscopic apparatus.

In order to achieve at least one object of the present invention, the present invention provides a control method for a magnetic control capsule endoscopic apparatus, comprising:
after the magnetic control capsule endoscopic apparatus is started, detecting a current motion mode of the magnetic control capsule endoscopic apparatus, where the motion mode comprises a vehicle-mounted mode and a normal mode, and a position of a magnetic ball in the vehicle-mounted mode is lower than the position of the magnetic ball in the normal mode;
keeping the position of the magnetic ball unchanged and directly entering an examination procedure when the motion mode is configured as the normal mode;
adjusting the position of the magnetic ball from a default position corresponding to the vehicle-mounted mode to a default position corresponding to the normal mode, and entering the examination procedure when the motion mode is configured as the vehicle-mounted mode.

In an embodiment of the present invention, after the examination procedure is finished, the method further comprises:
detecting the current motion mode of the magnetic control capsule endoscopic apparatus, and adjusting the position of the magnetic ball to a default position corresponding to the current motion mode before turning off the magnetic control capsule endoscopic apparatus.

In an embodiment of the present invention, after the magnetic control capsule endoscopic apparatus is started and before detecting the current motion mode of the magnetic control capsule endoscopic apparatus, the method further comprises:
detecting whether the magnetic control capsule endoscopic apparatus is normal;
driving the magnetic ball to a preset position to correct the position of the magnetic ball when the magnetic control capsule endoscopic apparatus is abnormal; and
detecting the current motion mode of the magnetic control capsule endoscopic apparatus when the magnetic control capsule endoscopic apparatus is normal.

In an embodiment of the present invention, the method further comprises:
constructing a virtual rectangular motion space corresponding to a motion range of the magnetic ball based on a maximum travel distance Lx of the magnetic ball in a horizontal direction, a maximum travel distance Lz in a vertical direction, and a maximum travel distance Ly in a direction perpendicular to a paper plane;
establishing a three-axis coordinate system with the center of the motion space as an origin, the horizontal rightward direction as a positive direction of an X axis, the vertical upward direction as a positive direction of a Z axis, and the direction perpendicular to the paper plane inwards as a positive direction of a Y axis; and
determining and adjusting the position of the magnetic ball with the three-axis coordinate system as a reference.

In an embodiment of the present invention, determining and adjusting the position of the magnetic ball with the three-axis coordinate system as the reference comprises:
establishing a corresponding relationship between the motion mode and the default position of the magnetic ball;
when the motion mode is the normal mode, coordinates of the default position of the magnetic ball corresponding to the motion mode are defined as (−Lx/2, −Ly/2, Lz/2); and
when the motion mode is the vehicle-mounted mode, the coordinates of the default position of the magnetic ball corresponding to the motion mode are defined as (0, −Ly/2, −Lz/2).

In order to achieve at least one object of the present invention, the present invention provides a control system for a magnetic control capsule endoscopic apparatus, the system comprises:
a monitoring module that is configured to detect a current motion mode of the magnetic control capsule endoscopic apparatus after the magnetic control capsule endoscope apparatus is started, where the motion mode comprises a vehicle-mounted mode and a normal mode, and a position of a magnetic ball in the vehicle-mounted mode is lower than the position of the magnetic ball in the normal mode; and
a processing module that is configured to keep the position of the magnetic ball unchanged and directly enter an examination procedure after the motion mode is configured as the normal mode; and
the processing module that is further configured to adjust the position of the magnetic ball from a default position corresponding to the vehicle-mounted mode to a default position corresponding to the normal mode, and enter the examination procedure, when the motion mode is configured as the vehicle-mounted mode.

In an embodiment of the present invention, the monitoring module is further configured to detect the current motion mode of the magnetic control capsule endoscopic apparatus after the examination procedure is finished; and
the processing module is further configured to adjust the position of the magnetic ball to a default position corresponding to the current motion mode before turning off the magnetic control capsule endoscopic apparatus.

In an embodiment of the present invention, the monitoring module is further configured to detect whether the magnetic control capsule endoscopic apparatus is normal after the magnetic control capsule endoscopic apparatus is started and before detecting the current motion mode of the magnetic control capsule endoscopic apparatus;
the processing module is further configured to drive the magnetic ball to a preset position to correct the position of the magnetic ball when the magnetic control capsule endoscopic apparatus is abnormal; and
the processing module is further configured to detect the current motion mode of the magnetic control capsule endoscopic apparatus when the magnetic control capsule endoscopic apparatus is normal.

In an embodiment of the present invention, the system further comprises: a coordinate system constructing module; where
the coordinate system constructing module is configured to construct a virtual rectangular motion space corresponding to a motion range of the magnetic ball based on a maximum travel distance Lx of the magnetic ball in a horizontal direction, a maximum travel distance Lz in a vertical direction, and a maximum travel distance Ly in a direction perpendicular to a paper plane;
the coordinate system constructing module is further configured to establish a three-axis coordinate system with the center of the motion space as an origin, the horizontal rightward direction as a positive direction of an X axis, the vertical upward direction as a positive direction of a Z axis, and the direction perpendicular to the paper plane inwards as a positive direction of a Y axis; and
the processing module is further configured to determine and adjust the position of the magnetic ball with the three-axis coordinate system as a reference.

In an embodiment of the present invention, the system further comprises: a mapping module; where
the mapping module is configured to establish a corresponding relationship between the motion mode and the default position of the magnetic ball;
coordinates of the default position of the magnetic ball corresponding to the motion mode are defined as (−Lx/2, −Ly/2, Lz/2) when the motion mode is the normal mode; and
the coordinates of the default position of the magnetic ball corresponding to the motion mode are defined as (0, −Ly/2, −Lz/2) when the motion mode is the vehicle-mounted mode.

The present invention has the following advantages over the prior art. According to the control method and the control system for the magnetic control capsule endoscopic apparatus, the position of the magnetic ball in the magnetic control capsule endoscopic apparatus is adjusted by identifying the normal mode and the vehicle-mounted mode, so that the magnetic control capsule endoscopic apparatus is suitable for a variety of application environments, and the safety performance of the magnetic control capsule endoscopic apparatus is improved.

### BRIEF DESCRIPTION OF THE DRAWINGS

FIG. 1 is a structural diagram of a magnetic control capsule endoscopic apparatus as described in the background of the present invention.

FIG. 2 is an exemplar process flow diagram of a method for the magnetic control capsule endoscopic apparatus, in accordance with an embodiment of the present invention.

FIG. 3 is a module diagram of a control system for the magnetic control capsule endoscope apparatus, in accordance with an embodiment of the present invention.

FIG. 4 is a module diagram of the control system for the magnetic control capsule endoscope apparatus, in accordance with another embodiment of the present invention.

### DETAILED DESCRIPTION

The present invention can be described in detail below with reference to the accompanying drawings and preferred embodiments. However, the embodiments are not intended to limit the present invention, and the structural, method, or functional changes made by those skilled in the art in accordance with the embodiments are included in the scope of the present invention.

Referring to FIG. 2, the present invention provides a control method for a magnetic control capsule endoscope apparatus in accordance with an embodiment of the present invention. The method comprises:
after the magnetic control capsule endoscopic apparatus is started, detecting a current motion mode of the magnetic control capsule endoscopic apparatus, where the motion mode comprises a vehicle-mounted mode and a normal mode, and a position of a magnetic ball in the vehicle-mounted mode is lower than the position of the magnetic ball in the normal mode; if the motion mode is configured as the normal mode, keeping the position of the magnetic ball unchanged, and directly entering an examination procedure; if the motion mode is configured as the vehicle-mounted mode, after adjusting the position of the magnetic ball from a default position corresponding to the vehicle-mounted mode to a default position corresponding to the normal mode, entering the examination procedure.

Further, after the examination procedure is finished, the method further comprises: detecting a current motion mode of the magnetic control capsule endoscopic apparatus, and adjusting the position of the magnetic ball to a default position corresponding to the current motion mode before turning off the magnetic control capsule endoscopic apparatus.

In an embodiment of the present invention, the magnetic control capsule endoscopic apparatus can be installed in a space of a stationary carrier or a space of a mobile carrier.

When the magnetic control capsule endoscope apparatus is installed in the space of the stationary carrier, it is generally configured to a normal mode. In the normal mode, either when the apparatus is not started, or when the apparatus is started but not yet entering the examination procedure, the position of the magnetic ball is typically high, which helps to prevent the magnetic ball from accidentally colliding with other objects and makes it easier for a subject to get on and off an examination bed of the magnetic control capsule endoscope apparatus.

When the magnetic control capsule endoscope apparatus is installed in the space of the mobile carrier, it is generally configured to a vehicle-mounted mode. In the vehicle-mounted mode, either when the apparatus is not started, or when the apparatus is started but not yet entering the examination procedure, the position of the magnetic ball is typically lower than it is in the normal mode, so that the center of gravity of the magnetic ball is lowered, which lowers the center of gravity of the entire magnetic control capsule endoscopic apparatus, and further improves the stability and the safety in the process of movement.

Further, the magnetic control capsule endoscopic apparatus may be in an abnormal state prior to being started during use due to unexpected power failure and shutdown, and the most obvious effect of the abnormal state is that the position of the magnetic ball is altered. Accordingly, in a preferred embodiment of the present invention, after the magnetic control capsule endoscopic apparatus is started and before detecting the current motion mode, the method further comprises: detecting whether the magnetic control capsule endoscopic apparatus is normal; if the magnetic control capsule endoscopic apparatus is abnormal, driving the magnetic ball to a preset position to correct the position of the magnetic ball; and if the magnetic control capsule endoscopic apparatus is normal, detecting the current motion mode, and entering the examination procedure according to the corresponding mode.

In a practical application, after detecting that the magnetic control capsule endoscopic apparatus is abnormal, the position of the magnetic ball can be corrected in the following way before entering the examination procedure. In one embodiment, the preset position is a position corresponding to the motion mode, and after the position of the magnetic ball is corrected according to the preset position, the examination procedure for the corresponding motion mode can be directly entered. If the preset position is the position of the magnetic ball in the normal mode, after the magnetic ball is driven to the position for correction, the examination procedure in the normal mode can be directly entered. If the preset position is the position of the magnetic ball in the vehicle-mounted mode, after the magnetic ball is driven to the position for correction, the examination procedure in the vehicle-mounted mode can be directly entered.

In another embodiment, if the preset position is any defined position other than the position corresponding to the motion mode, after the position of the magnetic ball is corrected according to the preset position, the magnetic ball is driven to the position corresponding to the motion mode, and the examination procedure for the corresponding motion mode can be entered. It is not be described in further detail herein.

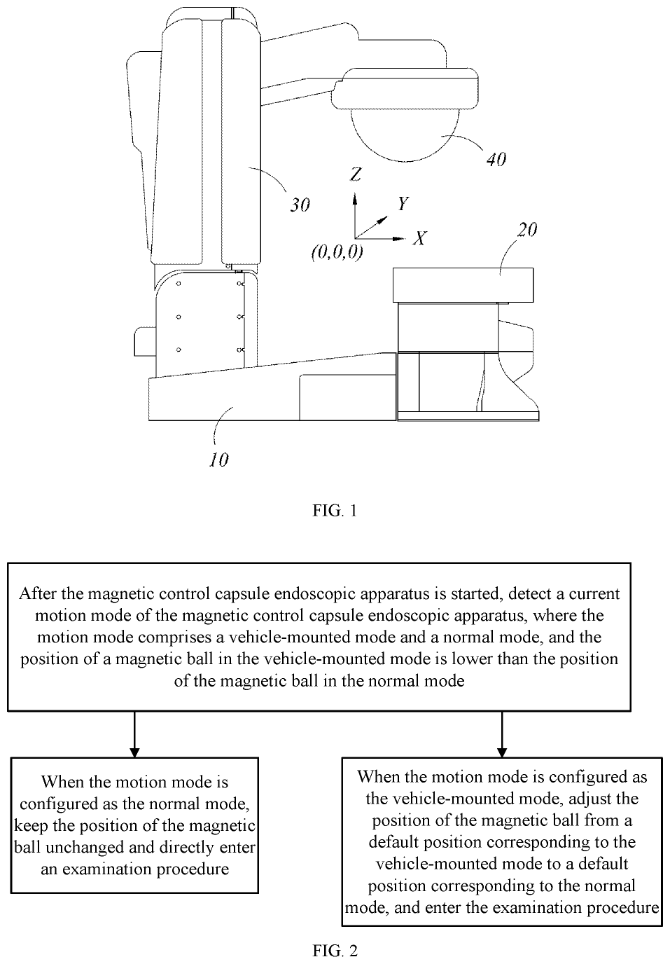

In a preferred embodiment of the present invention, the method further comprise: constructing a virtual rectangular motion space corresponding to a motion range of the magnetic ball based on a maximum travel distance Lx of the magnetic ball in the horizontal direction, a maximum travel distance Lz in the vertical direction, and a maximum travel distance Ly in the direction perpendicular to a paper plane, and the paper plane is an XZ plane formed by the horizontal direction and the vertical direction. Specifically, as shown in FIG. 1, a three-axis coordinate system is established with the center of the motion space as the origin, the horizontal rightward direction as the positive direction of the X axis, the vertical upward direction as the positive direction of the Z axis, and the direction perpendicular to the paper plane inwards as the positive direction of the Y axis, that is, the Y axis completes a right hand rule and is perpendicular to the X axis and the Z axis. The method further comprise: determining and adjusting the position of the magnetic ball with the three-axis coordinate system as a reference.

Preferably, the method further comprises: establishing a corresponding relationship between the motion mode and the default position of the magnetic ball. If the motion mode is the normal mode, coordinates of the default position of the magnetic ball corresponding to the motion mode are defined as (−Lx/2, −Ly/2, Lz/2); and if the motion mode is the vehicle-mounted mode, the coordinates of the default position of the magnetic ball corresponding to the motion mode are defined as (0, −Ly/2, −Lz/2).

Taking the motion mode as the vehicle-mounted mode as an example. Before entering the examination procedure, the position of the magnetic ball is adjusted in the following way: driving the magnetic ball to move a distance Lx/2 horizontally to the left along the X axis and a distance Lz vertically upward along the Z axis from its current position.

In a specific embodiment of the present invention, the value of Lx is taken to fluctuate within the range of 400 mm±20 mm, the value of Ly is taken to fluctuate within the range of 500 mm±20 mm, and the value of Lz is taken to fluctuate within the range of 300 mm±20 mm. For example, the motion range of the rectangular motion space is 400 mm*500 mm*300 mm, correspondingly, the coordinates of the default position of the magnetic ball are (−200, −250, 150) in the normal mode, and the coordinates of the default position of the magnetic ball are (0, −250, −150) in the vehicle-mounted mode.

In a practical application, in the normal mode, the coordinates of the default position of the magnetic ball correspond to the limit position of the X axis in the horizontal leftward direction, the limit position of the Y axis in the direction perpendicular to the paper plane outwards, and the limit position of the Z axis in the vertical upward direction; and in the vehicle-mounted mode, the coordinates of the default position of the magnetic ball correspond to the zero position of the X axis, the limit position of the Y axis in the direction perpendicular to the paper plane outwards, and the limit position of the Z axis in the vertical downward direction. Thus, in a specific embodiment of the present invention, a limit device may be disposed at the boundary intersection of the virtual rectangular motion space, so as to easily adjust the precise position of the magnetic ball, which is not further described herein.

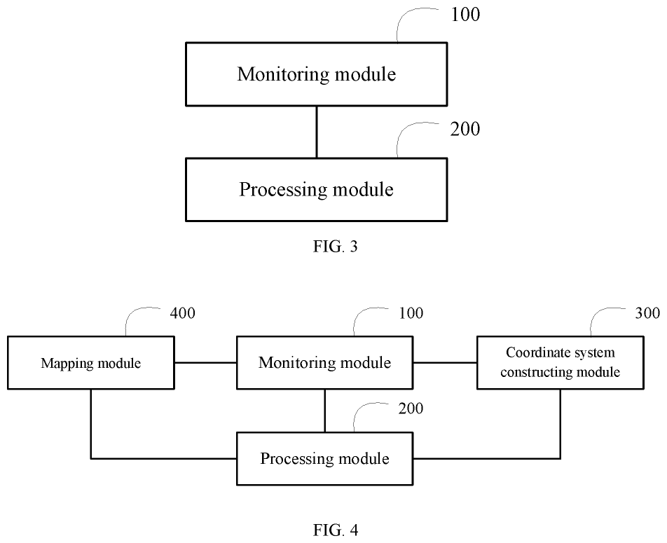

Referring to FIG. 3, a control system for a magnetic control capsule endoscopic apparatus is provided, in accordance with an embodiment of the present invention. The control system comprises a monitoring module 100 and a processing module 200.

The monitoring module 100 is configured to detect a current motion mode of the magnetic control capsule endoscope apparatus after the magnetic control capsule endoscope apparatus is started, where the motion mode comprises a vehicle-mounted mode and a normal mode, and a position of the magnetic ball in the vehicle-mounted mode is lower than that in the normal mode. The processing module 200 is configured to keep the position of the magnetic ball unchanged and directly enter an examination procedure when the motion mode is configured as the normal mode; and to firstly adjust the position of the magnetic ball from a default position corresponding to the vehicle-mounted mode to a default position corresponding to the normal mode, and to enter the examination procedure when the motion mode is configured as the vehicle-mounted mode.

Further, the monitoring module 100 is further configured for detecting a current motion mode of the magnetic control capsule endoscopic apparatus after the examination procedure is finished; and the processing module 200 is further configured for adjusting the position of the magnetic ball to a default position corresponding to the current motion mode before turning off the magnetic control capsule endoscopic apparatus.

Preferably, the monitoring module 100 is further configured for detecting whether the magnetic control capsule endoscopic apparatus is normal after the magnetic control capsule endoscopic apparatus is started and before detecting the current motion mode thereof; the processing module is further configured for driving the magnetic ball to a preset position to correct the position thereof if determining that the magnetic control capsule endoscopic apparatus is abnormal, and detecting the current motion mode of the magnetic control capsule endoscopic apparatus if determining that the magnetic control capsule endoscopic apparatus is normal.

Referring to FIG. 4, the system further comprises a coordinate system constructing module 300 and a mapping module 400.

The coordinate system constructing module 300 is configured for constructing a virtual rectangular motion space corresponding to a motion range of the magnetic ball based on a maximum travel distance Lx of the magnetic ball in the horizontal direction, a maximum travel distance Lz in the vertical direction, and a maximum travel distance Ly in the direction perpendicular to a paper plane, and the paper plane is an XZ plane formed by the horizontal direction and the vertical direction. Specifically, a three-axis coordinate system is established with the center of the motion space as the origin, the horizontal rightward direction as the positive direction of the X axis, the vertical upward direction as the positive direction of the Z axis, and the direction perpendicular to the paper plane inwards as the positive direction of the Y axis.

The processing module 200 is further configured for determining and adjusting the position of the magnetic ball with the three-axis coordinate system as a reference.

The mapping module 400 is configured for establishing a corresponding relationship between the motion mode and the default position of the magnetic ball. If the motion mode is the normal mode, coordinates of the default position of the magnetic ball corresponding to the motion mode are defined as (−Lx/2, −Ly/2, Lz/2); and if the motion mode is the vehicle-mounted mode, the coordinates of the default position of the magnetic ball corresponding to the motion mode are defined as (0, −Ly/2, −Lz/2).

Those skilled in the art can clearly understand that, for the convenience and conciseness of the description, the specific working process of the module described above is not repeated herein as it can be referred to the aforementioned method implementation.

In summary, the control method and control system for a magnetic control capsule endoscopic apparatus are provided, the position of the magnetic ball in the magnetic control capsule endoscopic apparatus is adjusted by identifying the normal mode and the vehicle-mounted mode, so that the magnetic control capsule endoscopic apparatus is suitable for a variety of application environments, and the safety performance of the magnetic control capsule endoscopic apparatus is improved.

It should be understood that, although the description is described in terms of embodiments, not every embodiment merely comprises an independent technical solution. Those skilled in the art should have the description as a whole, and the technical solutions in each embodiment may also be combined as appropriate to form other embodiments that can be understood by those skilled in the art.

The series of detailed descriptions set forth above are only specific descriptions of feasible embodiments of the present invention and are not intended to limit the scope of protection of the present invention. Equivalent embodiments or modifications made within the spirit of the present invention shall fall within the protection scope of the present invention.

1. A control method for a magnetic control capsule endoscopic apparatus, comprising:
after the magnetic control capsule endoscopic apparatus is started, detecting a current motion mode of the magnetic control capsule endoscopic apparatus, wherein the motion mode comprises a vehicle-mounted mode and a normal mode, and a position of a magnetic ball in the vehicle-mounted mode is lower than the position of the magnetic ball in the normal mode;
keeping the position of the magnetic ball unchanged and directly entering an examination procedure when the motion mode is configured as the normal mode;
adjusting the position of the magnetic ball from a default position corresponding to the vehicle-mounted mode to a default position corresponding to the normal mode, and entering the examination procedure when the motion mode is configured as the vehicle-mounted mode;
constructing a virtual rectangular motion space corresponding to a motion range of the magnetic ball based on a maximum travel distance Lx of the magnetic ball in a horizontal direction, a maximum travel distance Lz in a vertical direction, and a maximum travel distance Ly in a direction perpendicular to a paper plane;
establishing a three-axis coordinate system with the center of the motion space as an origin, the horizontal rightward direction as a positive direction of an X axis, the vertical upward direction as a positive direction of a Z axis, and the direction perpendicular to the paper plane inwards as a positive direction of a Y axis; and
determining and adjusting the position of the magnetic ball with the three-axis coordinate system as a reference;
wherein determining and adjusting the position of the magnetic ball with the three-axis coordinate system as the reference further comprises:
establishing a corresponding relationship between the motion mode and the default position of the magnetic ball;
when the motion mode is the normal mode, defining coordinates of the default position of the magnetic ball corresponding to the motion mode as (−Lx/2, −Ly/2, Lz/2); and
when the motion mode is the vehicle-mounted mode, defining the coordinates of the default position of the magnetic ball corresponding to the motion mode as (0, −Ly/2, −Lz/2).

2. The control method of claim 1, wherein after the examination procedure is finished, the method further comprises:
detecting the current motion mode of the magnetic control capsule endoscopic apparatus, and adjusting the position of the magnetic ball to a default position corresponding to the current motion mode before turning off the magnetic control capsule endoscopic apparatus.

3. The control method of claim 1, wherein after the magnetic control capsule endoscopic apparatus is started and before detecting the current motion mode of the magnetic control capsule endoscopic apparatus, the method further comprises:
detecting whether the magnetic control capsule endoscopic apparatus is normal;
driving the magnetic ball to a preset position to correct the position of the magnetic ball when the magnetic control capsule endoscopic apparatus is abnormal; and
detecting the current motion mode of the magnetic control capsule endoscopic apparatus when the magnetic control capsule endoscopic apparatus is normal.

4-5. (canceled)

6. A control system for a magnetic control capsule endoscopic apparatus, comprising:
one or more computer processors configured to:
a monitoring module that is configured to detect a current motion mode of the magnetic control capsule endoscopic apparatus after the magnetic control capsule endoscope apparatus is started, wherein the motion mode comprises a vehicle-mounted mode and a normal mode, and a position of a magnetic ball in the vehicle-mounted mode is lower than the position of the magnetic ball in the normal mode; and
a processing module that is configured to keep the position of the magnetic ball unchanged and directly enter an examination procedure after the motion mode is configured as the normal mode; and
the processing module that is further configured to adjust the position of the magnetic ball from a default position corresponding to the vehicle-mounted mode to a default position corresponding to the normal mode, and enter the examination procedure, when the motion mode is configured as the vehicle-mounted mode;
construct a virtual rectangular motion space corresponding to a motion range of the magnetic ball based on a maximum travel distance Lx of the magnetic ball in a horizontal direction, a maximum travel distance Lz in a vertical direction, and a maximum travel distance Ly in a direction perpendicular to a paper plane;
establish a three-axis coordinate system with the center of the motion space as an origin, the horizontal rightward direction as a positive direction of an X axis, the vertical upward direction as a positive direction of a Z axis, and the direction perpendicular to the paper plane inwards as a positive direction of a Y axis; and
determine and adjust the position of the magnetic ball with the three-axis coordinate system as a reference;
wherein the position of the magnetic ball with the three-axis coordinate system as the reference is determined and adjusted by the one or more computer processors being configured to:
establish a corresponding relationship between the motion mode and the default position of the magnetic ball;
define coordinates of the default position of the magnetic ball corresponding to the motion mode as (−Lx/2, −Ly/2, Lz/2) when the motion mode is the normal mode; and
define the coordinates of the default position of the magnetic ball corresponding to the motion mode as (0, −Ly/2, −Lz/2) when the motion mode is the vehicle-mounted mode.

7. The control system of claim 6, wherein one or more computer processors configured to:
the monitoring module is further configured to detect the current motion mode of the magnetic control capsule endoscopic apparatus after the examination procedure is finished; and
the processing module is further configured to adjust the position of the magnetic ball to a default position corresponding to the current motion mode before turning off the magnetic control capsule endoscopic apparatus.

8. The control system of claim 6, wherein one or more computer processors configured to:
the monitoring module is further configured to detect whether the magnetic control capsule endoscopic apparatus is normal after the magnetic control capsule endoscopic apparatus is started and before detecting the current motion mode of the magnetic control capsule endoscopic apparatus;
the processing module is further configured to drive the magnetic ball to a preset position to correct the position of the magnetic ball when the magnetic control capsule endoscopic apparatus is abnormal; and
the processing module is further configured to detect the current motion mode of the magnetic control capsule endoscopic apparatus when the magnetic control capsule endoscopic apparatus is normal.

9-10. (canceled)
